(19) United States
(12) Patent Application Publication
(10) Pub. No.: US 2026/0296994 A1
(43) Pub. Date: Oct. 1, 2026

(54) METHOD FOR PRODUCING FLUOROOLEFIN

(71) Applicant: KANTO DENKA KOGYO CO., LTD., Chiyoda-ku, Tokyo (JP)

(72) Inventors: Yuka SHIBUSAWA, Gunma (JP); Yukinobu SHIBUSAWA, Gunma (JP); Koyo KAWAI, Gunma (JP)

(21) Appl. No.: 18/996,543

(22) PCT Filed: Oct. 2, 2023
(86) PCT No.: PCT/JP2023/035831

§ 371 Date: Jan. 17, 2025

(30) Foreign Application Priority Data
Oct. 3, 2022 (JP) 2022-159262

(51) Int. Cl. C07C 17/25

(52) U.S. Cl. CPC C07C 17/25

(57) ABSTRACT

The purpose of the present invention is to provide a fluoroolefin production method, which has a high reaction conversion rate and selectivity of a target substance, and therefore is capable of obtaining the target substance in a high yield, has a low ratio of by-products, and generates no waste liquid, and solves the problems of the prior art. The method for producing a fluoroolefin by performing a dehydrofluorination reaction of a fluorinated alkane in the presence of activated carbon containing a metal salt.

### TECHNICAL FIELD

The present invention relates to a method for producing a fluoroolefin.

### BACKGROUND ART

Hydrofluoroolefin (HFO) is known as a desirable alternative to HCFC due to its low ODP (Ozone Depletion Potential) and GWP (Global Warming Potential) values. HFO is known as a useful compound such as a coolant, heat-transfer fluid, fire extinguishing agent, propellant, foaming agent, expansion agent, gas derivative, polymerization medium or monomer.

Patent Literature 1 describes a method for producing pentafluoropropene by contacting hexafluoropropane with a catalyst containing magnesium, zinc, aluminum, and the like, at a temperature of about 200° C. to 500° C., optionally in the presence of an inert gas. Patent Literature 2 describes a method for producing 1,1,1,3,3-pentafluoropropene, which is characterized by dehydrofluorinating 1,1,1,3,3,3-hexafluoropropane by bringing it into contact with activated carbon in a gaseous state. Patent Literature 3 describes a method for producing hydrofluoropropene, including: (a) introducing an alkali metal hydroxide feed stream into a reactor charged with halogenated propane represented by the formula: C₃FₓCl_{y}H_{8-x-y} (wherein x is 5 or 6, y is 0 or 1, and x+y≤6 is satisfied); (b) reacting, in a liquid phase, the halogenated propane with an aqueous base to form halogenated propene represented by the formula: C₃F_{z-1}H_{7-z} (wherein z is x−1); and (c) removing at least a portion of the halogenated propene from the reactor as a vapor product stream, wherein steps (a), (b), and (c) are at least partially performed simultaneously. Patent Literature 4 describes that 1,1,3,3,3-pentafluoropropene (CF₃CH═CF₂, HFC-1225zc) can be produced by pyrolyzing 1,1,1,3,3,3-hexafluoropropane (CF₃CH₂CF₃, HFC-236fa) in the absence of a dehydrofluorination catalyst at a temperature of from about 700° C. to about 1000° C. and a total pressure of about atmospheric pressure in an empty tubular reactor, the interior surfaces of which include materials of construction resistant to hydrogen fluoride.

### CITATION LIST

### Patent Literature

PTL 1: Japanese Translation of PCT International Application Publication No. 2001-509803
PTL 2: Japanese Patent Laid-Open No. H9-67281
PTL 3: U.S. Patent Publication No. 2011/0269999
PTL 4: Japanese Translation of PCT International Application Publication No. 2008-518938

### SUMMARY OF INVENTION

### Technical Problem

Disadvantageously, with regard to Patent Literature 1 and Patent Literature 2, the reaction conversion rate is low in the method using catalysts, containing magnesium, zinc, aluminum, and using coconut shell activated carbon. Also, disadvantageously, with regard to Patent Literature 3, in a reaction using an aqueous base, the reaction mode becomes complicated, such as continuously introducing an aqueous base and a phase transfer catalyst, and returning the unreacted raw material to the reactor under a reflux condition, etc., and moreover, under large scale production condition, a large amount of waste liquid is generated. Also, disadvantageously, with regard to Patent Literature 4, in the pyrolysis reaction under high temperature conditions, a plurality of impurities is synthesized with a high generation ratio, which have similar physical properties and are difficult to separate by distillation purification.

The purpose of the present invention is to provide a fluoroolefin production method. This method has a high reaction conversion rate and selectivity of a target substance, and therefore is capable of obtaining the target substance in a high yield, has a low ratio of by-products, and generates no waste liquid. Additionally, it solves the problems of the prior art.

### Solution to Problem

The present invention provides the following:

[1] A method for producing a fluoroolefin by performing a dehydrofluorination reaction of a fluorinated alkane in the presence of activated carbon containing a metal salt.
[2] The method according to [1], wherein the fluoroolefin is represented by the following general formula (1):

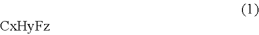

(where x=3-5, y≥1, z≥1, y+z≤2x).
[3] The method according to [2], wherein the fluorinated alkane is represented by the following general formula (2):

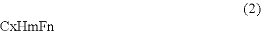

(where x=3-5, m≥2, n≥2, m+n=2x+2).
[4] The method according to any one of [1] to [3], wherein the dehydrofluorination reaction is performed in a gas phase.
[5] The method according to [4], wherein the dehydrofluorination reaction is performed at a temperature of 250 to 650° C.
[6] The method according to any one of [1] to [3], wherein the metal of the metal salt is an alkali metal and/or an alkaline earth metal.

### Advantageous Effects of Invention

According to the present invention, a fluoroolefin production method is provided. This method has a high reaction conversion rate and selectivity of a target substance, and therefore is capable of obtaining the target substance in a high yield, has a low ratio of by-products, and generates no waste liquid. Additionally, this method solves the problems of the prior art. According to the present invention, by using activated carbon containing a metal salt catalyst, a high conversion rate was achieved despite a reduction in the reaction temperature. As a result of lowering the reaction temperature, the formation of impurities with similar physical properties that are difficult to separate by distillation purification was suppressed. The conversion rate was high even at a temperature of about 500° C. in the reactor.

### BRIEF DESCRIPTION OF DRAWING

FIG. 1 is a schematic diagram of an example of the apparatus for performing a dehydrofluorination reaction of the present invention.

### DESCRIPTION OF EMBODIMENTS

### [Feedstock and Products]

The feedstock of the present invention is a fluorinated alkane composed of a fluorine atom, a hydrogen atom, and a carbon atom. In the present invention, the fluorinated alkane needs to have at least one hydrogen atom, since the fluorinated alkane produces fluoroolefin by the dehydrofluorination reaction.

The fluoroolefin of the product is preferably represented by the following general formula (1):

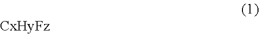

(where x=3-5, y≥1, z≥1, y+z≤2x).

In this case, the feedstock fluorinated alkane is preferably represented by the following general formula (2):

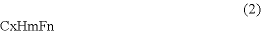

(where x=3-5, m≥2, n≥2, m+n=2x+2).

The fluorinated alkanes of the raw materials include, for example, 1,1,1,3,3,3-hexafluoropropane (HFC-236fa), 1,1,2,3,3,3-hexafluoropropane (HFC-236ea), 1,1,1,2,4,4,4-heptafluorobutane, 1,1,1,2,3,3,3-d heptafluoro-2-methylbutane, 1,1,1,3,3,3-heptafluoro-2-(monofluoromethyl) butane, 1,1,1,2,5,5,5-heptafluoropentane, 1,1,1,3,5,5,5-heptafluoropentane, and 1,1,1,4,4,4-hexafluorobutane, and these fluorinated alkanes may also be used alone or as mixtures of two or more.

The fluoroolefins of the product include, for example, 1,1,3,3,3-pentafluoropropene (HFO-1225zc), (E)-1,2,3,3,3-pentafluoropropene ((E)-HFO-1225ye), (Z)-1,2,3,3,3-pentafluoropropene ((Z)—HFO-1225ye), 1,1,1,4,4,4-hexafluorobut-2-ene, 3,3,3-trifluoro-2-trifluoromethylprop-1-ene, 1,1,1,5,5,5-hexafluoropent-2-ene, and 1,1,4,4,4-pentafluorobut-1-ene.

### [Reaction Catalyst]

The present invention uses activated carbon containing a metal salt as a reaction catalyst. Examples of the metal salt include halides (in particular, chlorides, fluorides, bromides, and iodides), hydroxides, carbonates, bicarbonates, nitrates, sulfites, sulfates, and acetates of alkali metals, alkaline earth metals, and the like. More specific examples of metal salts include, for example, alkali metal salts such as lithium chloride, sodium chloride, potassium chloride, lithium fluoride, sodium fluoride, potassium fluoride, lithium hydroxide, sodium hydroxide, potassium hydroxide, lithium carbonate, sodium carbonate, potassium carbonate, lithium bicarbonate, sodium bicarbonate, potassium bicarbonate, lithium sulfate, sodium sulfate, potassium sulfate, lithium acetate, sodium acetate, potassium acetate, and alkaline earth metal salts such as barium chloride, magnesium chloride, calcium chloride, barium fluoride, magnesium fluoride, calcium fluoride, barium hydroxide, magnesium hydroxide, calcium hydroxide, barium carbonate, magnesium carbonate, calcium carbonate, barium bicarbonate, barium bicarbonate, calcium bicarbonate, barium sulfate, magnesium sulfate, calcium sulfate, barium acetate, magnesium acetate, calcium acetate, and these may also be used alone or as mixtures of two or more.

The present invention features the activated carbon containing the metal salts described above. Examples of the activated carbon catalyst include catalysts for gas purification and coconut shell charcoals for catalyst/catalyst carrier (granular Shirasagi GX, SX, CX, XRC available from Takeda Pharmaceutical Co., Ltd., PCB available from Toyo Calgon Co., Ltd., activated carbon (coconut shell) available from Taihei Chemical Industrial Co., Ltd., and kuraraycoal GG, GC), and these can be used alone or in combination of two or more. The amount of the metal salt supported in the activated carbon is preferably 0.01 to 10 wt %, more preferably 0.1 to 5 wt %, and still more preferably 0.5 to 1.5 wt %, relative to the total weight of the supported catalyst. Activated carbon, which is not subjected to acid cleaning treatment, contains naturally occurring metal salts. In this case, the supported amount (i.e., content) of the metal salt is determined, for example, by SEM-EDX. The determination of the supported amount by SEM-EDX is performed by measuring several points on the sample surface using SEM-EDX and calculating the average value of those measurements. Preferred properties of the activated carbon include pH and acetone adsorption capacity. The preferred pH range is from 8 to 12, more preferably range is from 9 to 11. The preferred acetone adsorption capacity is preferably 29 wt % or more, more preferably 35 wt % or more. The shape of the activated carbon is not limiting, and any shape such as granule, powder, pellet, sphere, cylinder, honeycomb, sheet, or fiber can be adopted. The coal type of activated carbon is not limiting, and any of steam charcoal, crushed coal, granulated coal, and the like can be adopted which are derived from coconut shell charcoal, coal, charcoal, and the like. As the activated carbon, coconut shell charcoal is most preferable.

### [Reaction Conditions]

The reaction temperature is preferably 250° C. to 650° C., more preferably 400° C. to 625° C., still more preferably 500° C. to 600° C., particularly preferably 500° C. to 550° C. The contact time between the reactive substrate and the catalyst (metal salt supported activated carbon) is preferably 0.1 to 300 seconds, more preferably 5 to 120 seconds, still more preferably 10 to 60 seconds.

### [Carrier Gas]

In performing the present invention, a carrier gas is used for dilution of the raw material gas, drying of the reactor, and the like. In particular, substances such as raw material and reaction product can be moved in the reactor while the concentration is controlled by the flow of the carrier gas. As the carrier gas is selected a gas that reacts with none of these substances. They include, for example, nitrogen, and rare gases (helium, neon, argon, etc.). When using the carrier gas, the carrier gas is usually mixed at a ratio of 0 to 99%, more preferably 0 to 75%, and most preferably 0 to 50% for the total flow rate of the substance such as raw material and reaction product.

### [Reaction Apparatus]

The material of a reaction apparatus includes, for example, corrosion resistant metals such as stainless steel, Inconel, Monel, HASTELLOY, and nickel. Among these, nickel is preferred in terms of corrosion resistance.

An example of the apparatus for performing a dehydrofluorination reaction of the present invention is a cylindrical tube equipped with a heater for controlling the reaction temperature, which is configured to fill any of catalysts with various shapes therein, and flow the raw material gas from one end of the tube to the other. Regarding the raw material gas flow direction, when the cylindrical tube filled with the catalyst is extended vertically, it is preferable for the raw material gas to flow downward gradually and uniformly, as this allows the raw material gas to flow gradually by using gravity. When the cylindrical tube is extended vertically and the raw material gas flows upward, it is desirable in terms of reaction efficiency to dispose a pellet shaped catalyst with a large particle size at the lower section of the cylindrical tube and dispose a powder shaped catalyst with a small particle size at the upper section of the cylindrical tube.

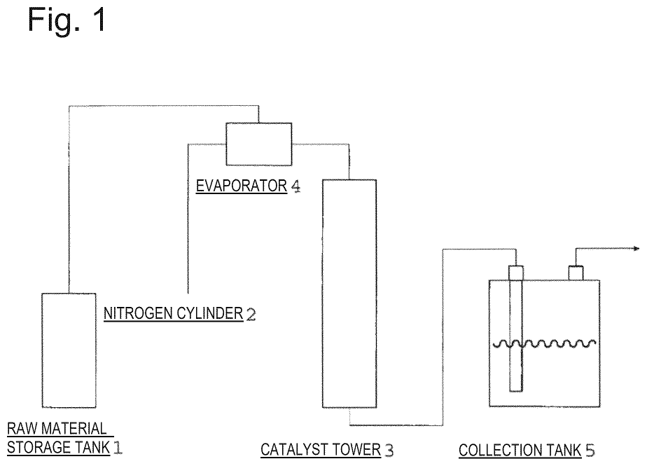

In FIG. 1, which schematically illustrates a specific example of the apparatus for performing a dehydrofluorination reaction of the present invention, the apparatus is composed of a raw material storage tank 1 containing a raw material (reaction substrate), a gas cylinder (nitrogen cylinder 2 in FIG. 1) that supplies a carrier gas (e.g. nitrogen gas) to an evaporator 4, a cylindrical catalyst tower 3 connected with the raw material storage tank and piping and installed vertically, an evaporator 4 installed just before the catalyst tower, and a collection tank 5 located downstream of the catalyst tower. When the raw material is liquid at room temperature, it is injected as a liquid by a syringe pump (not shown) into the evaporator 4, which is heated to a temperature higher than the boiling point of the raw material, and vaporized by the evaporator 4. The raw material gas mixes with the carrier gas (nitrogen gas) in the evaporator 4, and the mixed gas comes into contact with the catalyst as it moves from the top to the bottom of the catalyst tower, and the dehydrofluorination reaction is facilitated. While the reaction gas, which has passed through the bottom of the catalyst tower, moves through a collection tank filled with water inside, the reaction product, unreacted raw material, and hydrogen fluoride are collected in the collection tank, while only the carrier gas is discharged from the reactor.

### EXAMPLES

### [Preparation of Catalyst]

The catalyst used in the examples was prepared by immersing coconut shell based activated carbon supplied by Osaka Gas Chemicals Co., LTD., granular Shirasagi C2X (grade name: C2x4/6-2) in an aqueous solution containing the metal salt described in Table 1. The amount supported in the catalyst was determined using SEM-EDX to confirm the concentration distribution of the element on the surface of the measuring object. The supported amount was determined by measuring the sample surface at three points and averaging the measured values.

### Examples 1 to 13

Activated carbon containing the metal salt described in Table 1 was filled into a cylindrical reactor made of nickel (size: 2 inches in diameter, 1000 mm in length), and dried under heat, and then the raw material 1,1,1,3,3,3-hexafluoropropane (HFC-236fa) was passed from the top end of the cylindrical reactor to the bottom end without using carrier gas, and reacted at the temperature, linear speed, and residence time described in Table 1. In the post stage of the reactor, a water trap for removing the generated HF, a molecular sieve (MS-3A) packed tower for dehydration treatment of the crude product gas, and a filter for removing particles were used to treat the resulting product and give the crude product gas. The composition of the crude product gas was confirmed by GC analysis. The results are shown in Table 1.

### Comparative Examples 1 to 3

Activated carbon not supporting a metal salt was used as a catalyst, or a reaction was performed in the same manner as in Example 1 without using a catalyst. The results are shown in Table 1.

TABLE 1

| | CATALYST (ACTIVATED CARBON) | | | | | | | |
|---|---|---|---|---|---|---|---|---|
| | AMOUNT | | | | | | | |
| | OF METAL | | | CARRIER | | | | |
| | SALT | | | (ACTIVATED | FILLING | INTERNAL | LINEAR | RESIDENCE |
| | SUPPORTED | METAL | COUNTER | CARBON) | AMOUNT | TEMPERATURE | SPEED | TIME |
| UNIT | % | ION | ION | GRADE | g | ° C. | cm/s | s |
| EXAMPLE 1 | 1 | K | OH | C2x4/6-2 | 161 | 550 | 0.9 | 56 |
| EXAMPLE 2 | 1 | K | CO3 | C2x4/6-2 | 160 | 550 | 0.9 | 56 |
| EXAMPLE 3 | 1 | K | CH3COO | C2x4/6-2 | 164 | 500 | 0.9 | 56 |
| EXAMPLE 4 | 1 | K | HCO3 | C2x4/6-2 | 158 | 500 | 0.9 | 56 |
| EXAMPLE 5 | 2 | K | OH | C2x4/6-2 | 162 | 500 | 0.9 | 56 |
| EXAMPLE 6 | 5 | K | OH | C2x4/6-2 | 169 | 500 | 0.9 | 56 |
| EXAMPLE 7 | 2 | K | CO3 | C2x4/6-2 | 160 | 500 | 0.9 | 56 |
| EXAMPLE 8 | 0.5 | Li | OH | C2x4/6-2 | 164 | 500 | 0.9 | 56 |
| EXAMPLE 9 | 1 | Na | OH | C2x4/6-2 | 164 | 500 | 0.9 | 56 |
| EXAMPLE 10 | 0.5 | Mg | Cl | C2x4/6-2 | 160 | 500 | 0.9 | 56 |
| EXAMPLE 11 | 1 | Mg | Cl | C2x4/6-2 | 160 | 500 | 0.9 | 56 |
| EXAMPLE 12 | 0.5 | Ca | Cl | C2x4/6-2 | 160 | 500 | 0.9 | 56 |
| EXAMPLE 13 | 1 | Ca | Cl | C2x4/6-2 | 160 | 500 | 0.9 | 56 |
| COMPARATIVE | NONE | NONE | NONE | C2x4/6-2 | 158 | 500 | 0.8 | 59 |
| EXAMPLE I | | | | | | | | |
| COMPARATIVE | NONE | NONE | NONE | NONE | — | 550 | 0.7 | 72 |
| EXAMPLE 2 | | | | | | | | |
| COMPARATIVE | NONE | NONE | NONE | NONE | — | 775 | 2.1 | 45 |
| EXAMPLE 3 | | | | | | | | |

| | | IMPURITY GENERATION | | | |
|---|---|---|---|---|---|
| | CONVERSION | RATIO | | | |
| | RATE | 1225yeZ | 1225yeE | 1234zeE | SELECTIVITY |
| UNIT | % | % | % | % | % |
| EXAMPLE 1 | 89.8 | 0.0650 | 0.0168 | 0.0019 | 85.6 |
| EXAMPLE 2 | 91.3 | 0.0887 | 0.0232 | 0.0024 | 82.8 |
| EXAMPLE 3 | 86.2 | 0.0855 | 0.0212 | 0.0021 | 84.7 |
| EXAMPLE 4 | 87.2 | 0.0589 | 0.0152 | 0.0028 | 89.1 |
| EXAMPLE 5 | 86.5 | 0.0675 | 0.0170 | 0.0021 | 87.0 |
| EXAMPLE 6 | 81.5 | 0.0744 | 0.0184 | 0.0028 | 88.5 |
| EXAMPLE 7 | 85.6 | 0.0807 | 0.0199 | 0.0023 | 86.1 |
| EXAMPLE 8 | 68.9 | 0.0410 | 0.0117 | 0.0047 | 86.8 |
| EXAMPLE 9 | 71.2 | 0.0410 | 0.0123 | 0.0056 | 87.4 |
| EXAMPLE 10 | 56.5 | 0.0474 | 0.0146 | 0.0037 | 88.7 |
| EXAMPLE 11 | 61.9 | 0.0588 | 0.0173 | 0.0121 | 89.4 |
| EXAMPLE 12 | 61.5 | 0.0360 | 0.0115 | 0.0033 | 86.7 |
| EXAMPLE 13 | 62.5 | 0.0380 | 0.0116 | 0.0033 | 87.6 |
| COMPARATIVE | 38.5 | 0.0159 | 0.0049 | 0.0030 | 96.6 |
| EXAMPLE I | | | | | |
| COMPARATIVE | 37.2 | 0.0255 | 0.0079 | 0.0238 | 97.4 |
| EXAMPLE 2 | | | | | |
| COMPARATIVE | 73.0 | 0.3959 | 0.1061 | 0.1075 | 94.4 |
| EXAMPLE 3 | | | | | |

In Table 1, the impurity generation ratio is the GC area ratio (corresponding to the molar ratio) of each impurity when the GC area of the total crude product injected into the gas chromatograph (GC) is 100%. The conversion rate (%) is 100%−[(post reaction: molar number of HFC-236fa in crude gas)/(pre reaction: molar number of introduced HFC-236fa)]×100%. The selectivity (%) is [(post reaction: molar number of HFO-1225zc in crude gas/molar number of converted HFC-236fa)]×100%. In addition, the indication of impurities is as follows.
1225yeZ: Z isomer of CF₃—CF═CHF
1225yeE: E isomer of CF₃—CF═CHF
1234zeE: E isomer of CF₃—CH═CHF

From Table 1, the dehydrofluorination reaction of 1,1,1,3,3,3-hexafluoropropane (HFC-236fa) was conducted at a temperature of 500 to 550° C. in the presence of a metal salt-supported activated carbon containing 0.5 to 5 wt % of alkali metal hydroxide, alkali metal bicarbonate, alkali metal carbonate, alkali metal acetate, or alkaline earth metal chloride. When using alkali metal salts (Examples 1 to 9), 1,1,3,3,3-pentafluoropropene (HFO-1225zc) was obtained with a conversion rate exceeding 60% and a selectivity exceeding 80%. When using alkaline earth metal salts (Examples 10 to 13), the same product was obtained with a conversion rate exceeding 56% and a selectivity exceeding 86%. In terms of obtaining high purity of HFO-1225zc excluding impurities, a post purification yield of 60% or more was achieved in Examples 1 to 9, and a post purification yield of 50% or more was achieved in Examples 10 to 13. When alkaline earth metal salt was used as metal salt, the conversion rate was lower than when alkali metal salt was used, but the selectivity was about the same as when alkali metal salt was used. On the other hand, Comparative Example 1, which used activated carbon without a metal salt as a catalyst, and Comparative Example 2, which used no catalyst, gave conversion rates of 38.5% and 37.2%, respectively. In addition, when no catalyst was used but the reaction temperature was as high as 775° C., a conversion rate of 73% was obtained. However, impurities (1225yeZ, 1225yeE, 1234zeE) difficult to separate from the product HFO-1225zc were generated at 0.1% or more each. The production conditions that generate these impurities in a concentration of 0.1% or more cannot be adopted because the impurities cannot be removed by distillation purification.

1. A method for producing a fluoroolefin by performing a dehydrofluorination reaction of a fluorinated alkane in the presence of activated carbon containing a metal salt.

2. The method according to claim 1, wherein the fluoroolefin is represented by the following general formula (1): 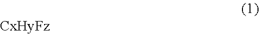
(where x=3-5, y≥1, z≥1, y+z≤2x).

3. The method according to claim 2, wherein the fluorinated alkane is represented by the following general formula (2): 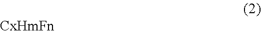
(where x=3-5, m≥2, n≥2, m+2, m+n=2x+2).

4. The method according to claim 3, wherein the fluoroolefin is 1,1,3,3,3-pentafluoropropene (HFO-1225zc), and the fluorinated alkane is 1,1,1,3,3,3-hexafluoropropane (HFC-236fa).

5. The method according to claim 1, wherein the dehydrofluorination reaction is performed in a gas phase.

6. The method according to claim 5, wherein the dehydrofluorination reaction is performed at a temperature of 250 to 650° C.

7. The method according to claim 6, wherein the dehydrofluorination reaction is performed at a temperature of 400 to 625° C.

8. The method according to claim 7, wherein the dehydrofluorination reaction is performed at a temperature of 500 to 550° C.

9. The method according to claim 1, wherein the metal of the metal salt is an alkali metal and/or an alkaline earth metal.

10. The method according to claim 9, wherein the amount of the metal salt contained in the activated carbon is 0.5 to 5 wt %.

11. The method according to claim 4, wherein the metal of the metal salt is an alkali metal.

12. The method according to claim 11, wherein the amount of the metal salt contained in the activated carbon is 0.5 to 5 wt %.

13. The method according to claim 12, wherein the dehydrofluorination reaction is performed in a gas phase.

14. The method according to claim 13, wherein the dehydrofluorination reaction is performed at a temperature of 500 to 550° C.
